SEQUENCE LISTING (20 sequences)

SEQ ID NO: 1 - DNA, 43 bp, human
  promoter (1)...(7)
  misc_RNA (8)...(30): n=a,t,g,c
ggacgagnnnnnnnnnnnnnnnnnnnnnnnaattcatctatgt

SEQ ID NO: 2 - DNA, 43 bp, human
  misc_RNA (12)...(34): n=a,t,g,c
cctgctcctagnnnnnnnnnnnnnnnnnnnnnnngtagataca

SEQ ID NO: 3 - DNA, 59 bp, human
  misc_RNA (7)...(53)
gatccggatgatctggatccaccaattcaagagattggtggatccagatcatctttttg

SEQ ID NO: 4 - DNA, 59 bp, human
  misc_RNA (3)...(59)
gcctactagacctaggtggttaagttctctaaccacctaggtctagtagaaaaacttaa

SEQ ID NO: 5 - DNA, 75 bp, human
  misc_RNA (7)...(69)
gatccgggtgatctggatctaccaaggcttgttttcaagagaaacaagtcttggtggatccagatcatctttttg

SEQ ID NO: 6 - DNA, 69 bp, human
  misc_RNA (3)...(59)
gcccactagacctagatggttcgaaagttctctttgttcagaaccactaggtctagtagaaaaacttaa

SEQ ID NO: 7 - DNA, 30 bp, human
  unsure (9)...(21): n=a,t,g,c
tgggaaaannnnnnnnnnnnntttttagag

SEQ ID NO: 8 - DNA, 30 bp, human
  unsure (13)...(25): n=a,t,g,c
accctttttttcnnnnnnnnnnnnnatctc

SEQ ID NO: 9 - DNA, 31 bp, human
  misc_RNA (6)...(31): Viral vector sequence
aaaggatgatctggatccaccaagacttgtt

SEQ ID NO: 10 - DNA, 31 bp, human
  misc_RNA (2)...(27): Viral vector sequence
ctactagacctaggtggttctgaacaaaaaa

SEQ ID NO: 11 - DNA, 59 bp, human
  misc_RNA (17)...(42): n=a,t,g,c
agcagaagactaaaagnnnnnnnnnnnnnnnnnnnnnnnnnnttttatgtcttctacga

SEQ ID NO: 12 - DNA, 19 bp, human
  primer_bind (11)...(19): Viral vector sequence
acgcaggtgtagcagaaga

SEQ ID NO: 13 - DNA, 59 bp, human; identical to SEQ ID NO: 11
  misc_RNA (17)...(42): n=a,t,g,c

SEQ ID NO: 14 - DNA, 20 bp, human
  primer_bind (1)...(11): Viral vector sequence
cagaagatgctcacgacgct

SEQ ID NO: 15 - DNA, 78 bp, human
  misc_RNA (27)...(52): n=a,t,g,c
acgcaggtgtagcagaagactaaaagnnnnnnnnnnnnnnnnnnnnnnnnnnttttatgtcttctacgagtgctgcga

SEQ ID NO: 16 - DNA, 78 bp, human
  misc_RNA (27)...(52): n=a,t,g,c
tgcgtccacatcgtcttctgattttcnnnnnnnnnnnnnnnnnnnnnnnnnnaaaatacagaagatgctcacgacgct

SEQ ID NO: 17 - DNA, 30 bp, human
  misc_RNA (5)...(30): n=a,t,g,c
aaagnnnnnnnnnnnnnnnnnnnnnnnnnn

SEQ ID NO: 18 - DNA, 30 bp, human
  misc_RNA (1)...(26): n=a,t,g,c
nnnnnnnnnnnnnnnnnnnnnnnnnnaaaa

SEQ ID NO: 19 - DNA, 44 bp, human
  mRNA (10)...(35): n=a,t,g,c
tgggaaaagnnnnnnnnnnnnnnnnnnnnnnnnnntttttagag

SEQ ID NO: 20 - DNA, 44 bp, human
  misc_RNA (10)...(35): n=a,t,g,c
acccttttcnnnnnnnnnnnnnnnnnnnnnnnnnnaaaaatctc

(19) United States
(12) Patent Application Publication
(10) Pub. No.: US 2005/0074889 A1
(43) Pub. Date: Apr. 7, 2005

(54) Methods for gene function analysis

(75) Inventors: Peter Chumakov, Solon, OH (US); Alex Chenchik, Emerald Hills, CA (US)

Correspondence Address: MOSER, PATTERSON & SHERIDAN, L.L.P., Suite 1500, 3040 Post Oak Blvd., Houston, TX, 77056, US

(73) Assignee: System Biosciences LLC.

(21) Appl. No.: 10/766,605

(22) Filed: Jan. 27, 2004

Related U.S. Application Data
(62) Division of application No. 10/658,632, filed on Sep. 8, 2003.

(51) Int. Cl. C12N 15/867; C12N 7/01

(52) U.S. Cl. 435/456; 435/235.1

(57) ABSTRACT

The present invention provides methods for analysis of gene function using effector libraries.

This is a divisional of copending application Ser. No.10/658,632, filed on Sep. 8, 2003.

### BACKGROUND OF THE INVENTION

As a result of various genome-wide sequencing projects such as the Human Genome Project, researchers now know the sequence of many human genes. However, there is an urgent need to develop tools to uncover the function(s) of each of these genes. Importantly, functional genomic studies will speed up the discovery and validation of drug targets.

Several new technologies have been developed recently for selective inactivation of gene products in vivo, including catalytic RNAs such as ribozymes, maxizymes and aptazymes (see, e.g., Kuwabara, T. et al., Trends Biotechnol., 18:462-468 (2000); and Famulok, M., and Verma, S., Trends Biotechnol., 20 462-468 (2002)); protein-binding RNA motifs such as aptamers and intramers (again, see, e.g., Kuwabara, T. et al., Trends Biotechnol., 18:462-468 (2000)), and genetic suppressor elements (see, e.g., Robinson, I. B., and Gudkov, A. V, Methods in Molecular Biology, Tumor Suppressor Genes: Pathways and Isolation Strategies (Ed. Wafik, S. E.) Humana Press Inc., 222:411-434 (2002)). However, where antisense (both RNA and deoxyoligonucleotides) and genetic suppressor elements have been used commonly for high-throughput gene function analysis; neither catalytic RNAs, aptamers or intramers have found broad application in such analyses.

RNAi is the sequence-specific, post-transcriptional silencing of a gene's expression by double-stranded RNA. RNAi is mediated by 21- to 25-nucleotide, double-stranded RNA molecules referred to as small interfering RNAs (siRNAs) that are derived by enzymatic cleavage of long, double-stranded RNA in cells. siRNAs also can be synthesized chemically or enzymatically outside of cells and subsequently delivered to cells (see, for example, Fire, et al., Nature, 391:806-11 (1998); Tuschl, et al., Genes and Dev., 13:3191-97 (1999); and Elbashir, et al., Nature, 411:494-498 (2001)).

Double stranded siRNAs mediate gene silencing by targeting for disruption or cleavage messenger RNAs (mRNAs) that contain the sequence of one strand of the siRNA. Short (19-29 nucleotide length) siRNAs introduced into mammalian cells mediate sequence-specific gene silencing, whereas long, double-stranded RNA (more than about 30 nucleotides) has been shown to induce non-specific responses, such as interferon response.

Thus, siRNA can be used for selective inactivation (silencing) of gene products. The typical approach in using siRNA is to study the effect of various siRNAs on each target gene; that is, performing a functional screen by inactivating one gene at a time. Several companies (Dharmacon, Qiagen, and Ambion, for example) currently offer custom siRNA synthesis services that can be used in such gene knockdown experiments. In addition, progress has been made to overcome the transient nature of the gene silencing effects of synthetic siRNAs as several expression plasmid and retroviral vectors have been developed that provide continuous siRNA expression. These vectors direct the synthesis of fold-back stem-loop transcripts (hairpin siRNAs) from an RNA polymerase III promoter (U6 or H1), where the hairpin structure subsequently is converted into a non-hairpin double-stranded siRNA structure after intracellular processing.

However, the need remains for a process and system for efficiently and effectively performing functional analysis on many genes simultaneously. The present invention satisfies this need in the art.

### SUMMARY OF THE INVENTION

The present invention is directed to an innovative, rapid and robust procedure and system for performing functional analysis on many genes simultaneously. Specifically, the present invention is directed to embodiments of an effector library that 1) provides high transfection efficiency, and, in some embodiments induces gene silencing in most cell lines; 2) allows selection of transfected cells by a variety of selection markers or reporter constructs; and 3) provides inducible expression of the effector products.

Thus, the present invention provides in one embodiment a method for identification of sequences that affect gene function comprising: obtaining at least two sequence-defined, substantially identical sets of at least 100 effector nucleic acid sequences, where one of the at least two sets of effector nucleic acid sequences is arrayed on a microarray; cloning another of the at least two effector nucleic acid sets into a viral expression vector to produce effector constructs; packaging the effector constructs into viral particles to produce a viral effector library; transducing target cells with the viral effector library; assaying the target cells for a characteristic of interest; selecting one or more target cells with the phenotype of interest; purifying effector nucleic acid from the target cell with the phenotype of interest; hybridizing the purified effector nucleic acid to the microarray; and identifying an effector sequence present in the target cell with the phenotype of interest.

In another embodiment of the present invention, there is provided a viral effector library packaged in viral particles consisting essentially of: viral vectors; at least 100 sequence-specific, heterologous nucleic acid sequences inserted into the viral vectors, where the at least 100 heterologous effector nucleic acid sequences are substantially the same as or substantially complementary to probes on a nucleic acid microarray; and one or more eukaryotic promoters operably linked to the heterologous nucleic acid sequences.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

FIG. 1 is a simplified flow chart of one embodiment of a method according to the present invention.

FIG. 2 is a schematic of a general design of construction of a viral effector (here, siRNA) library (step 200 of FIG. 1).

FIG. 3 is a schematic of one embodiment of the double promoter lentiviral vector of the present invention.

FIG. 4 is a schematic showing the construction of an effector library, the content of which can be verified by, for example, hybridization to a microarray.

FIG. 5 is a schematic of a general design of one embodiment of a method (100) according to the present invention using an effector library, showing transduction (step 300 of FIG. 1), selection (step 400 of FIG. 1), and effector gene identification/gene function analysis (step 500 of FIG. 1).

FIG. 6 is a schematic showing parallel construction of an effector library (again siRNA) and a reporter vector.

FIG. 7 is a schematic showing a functional screen using both an effector library and a reporter vector.

FIG. 8 shows one embodiment of an a lentiviral reporter vector according to the present invention.

FIG. 9 illustrates a design and cloning method for a single promoter effector cassette (here, siRNA), according to one embodiment of the present invention.

FIG. 10 illustrates a design and cloning method for a double promoter effector cassette (again, siRNA), according to one embodiment of the present invention.

FIG. 11 is a schematic representation of the repression and activation of effector expression in a tet/on tet/off system.

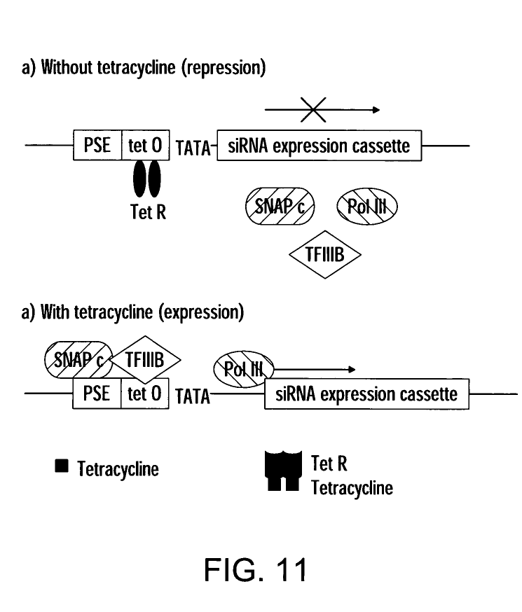

FIG. 12 illustrates the conversion of a single stranded effector oligo template into double stranded cassette by PCR, generation of sticky ends by restriction enzyme digestion, and ligation of the double-stranded cassette effector cassette into an expression vector.

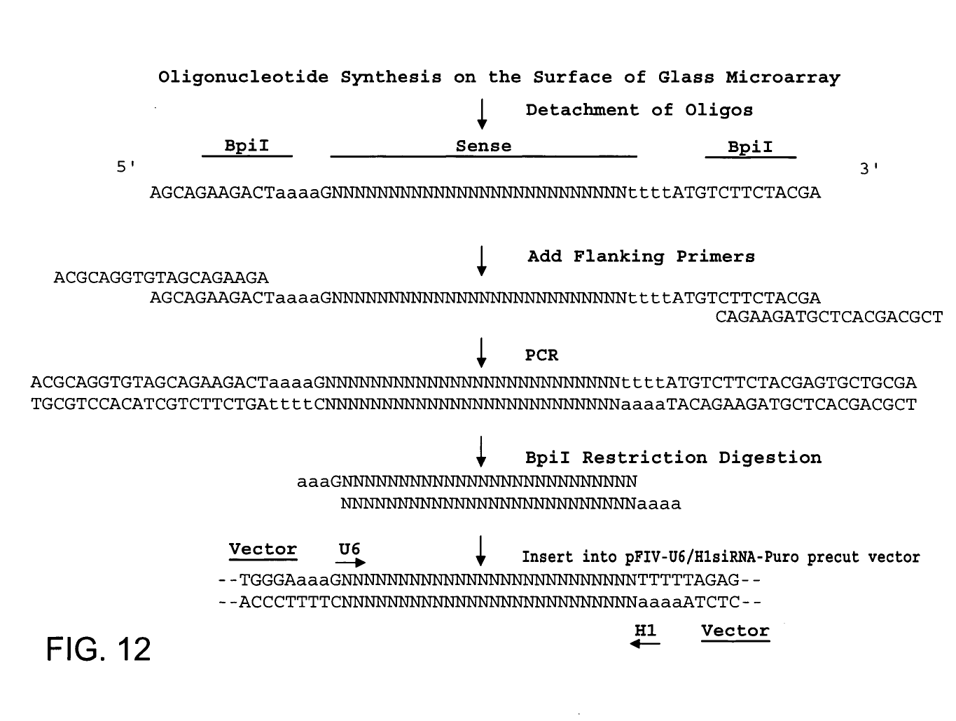

### DETAILED DESCRIPTION

In the following description, numerous specific details are set forth to provide a more thorough understanding of the present invention. However, it will be apparent to one of skill in the art that the present invention may be practiced without one or more of these specific details. In other instances, well-known features have not been described in order to avoid obscuring the present invention.

The present invention is directed to an innovative, rapid and robust procedure and system to perform functional analysis on many genes simultaneously. Specifically, the present invention is directed to embodiments of viral effector libraries that 1) provide high transfection efficiency, which, in some embodiments, induce arbitrary gene-specific silencing in most cell lines, 2) allow selection of transfected cells by a variety of selection markers or reporter assays in vitro and in vivo for variety of diseased and normal biological systems, and 3) provide efficient and inducible expression of the effector products.

Generally, conventional methods of molecular biology, microbiology, recombinant DNA techniques, cell biology, virology within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); and RNA Viruses: A practical Approach, (Alan, J. Cann, Ed., Oxford University Press, 2000).

A “vector” is a replicon, such as plasmid, phage, retroviral construct or cosmid, to which another DNA segment may be attached. Vectors are used to transduce and express the DNA segment in cells.

An “effector” is a biochemical molecule that can effect the transcription, translation, expression or function of another molecule such as a target gene or the product of a target gene. Effectors may be full-length proteins, protein domains, peptides, single-stranded or double-stranded deoxy- or ribo-oligonucleotides, siRNAs or their mimetics or analogues.

A “promoter sequence” is a DNA regulatory region capable of binding RNA polymerase in a cell and initiating transcription of a coding sequence. For example, the promoter sequence may be bounded at its 3′ terminus by the transcription initiation site and extend upstream (in the 5′ direction) to include the minimum number of bases or elements necessary to initiate transcription at levels detectable above background. Within the promoter sequence may be found a transcription initiation site, as well as protein binding domains responsible for the binding of RNA polymerase or other transcriptional factors. Eukaryotic promoters will often, but not always, contain “TATA” boxes and “CAT” boxes. Various promoters, such as those recognized by RNA polymerase II or RNA polymerase III and/or inducible promoters known in the art may be used to drive the various vectors of the present invention.

The terms “restriction endonucleases” or “restriction enzymes” refer generally to bacterial enzymes that cut double-stranded DNA at or near a specific nucleotide sequence.

A cell has been “transformed”, “transduced” or “transfected” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a host cell chromosome or is maintained extra-chromosomally so that the transforming DNA is inherited by daughter cells during cell replication. Such a stably transformed eukaryotic cell is able to establish cell lines or clones comprised of a population of daughter cells containing the transforming DNA.

The term “small interfering RNA” or “siRNA” refers generally to substantially double-stranded RNA molecules that inhibit the expression of a gene with which they share homology.

The term “microarray” refers to arrays or ordered arrangements of different targets, such as proteins, peptides or nucleic acids on a solid or semi-solid support such as a slide, membrane, chip, bead, or microwell plate with a known location or address of each target. Targets can be bound to a support by photolithographic techniques, phosphoramidite chemistry, photochemistry, electrochemistry, covalent or non-covalent immobilization or other methods known in the art.

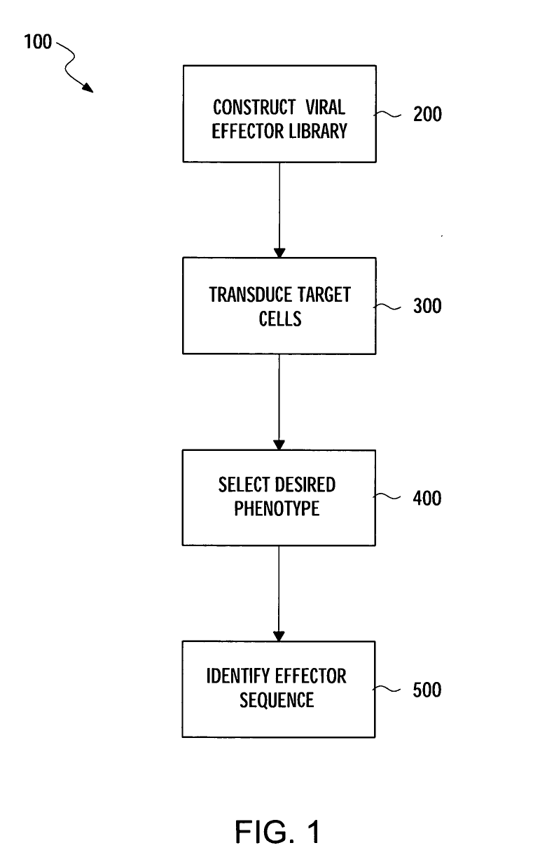

FIG. 1 is a block diagram of a simplified method 100 according to one embodiment of the present invention. In a first step, a viral effector library is constructed 200. Next, target cells of interest are transduced with the viral effector library (step 300) and target cells having a pre-determined phenotype are selected (step 400). Finally, at step 500, analysis is conducted by analyzing the target cells to identify the effector sequence responsible for the phenotype.

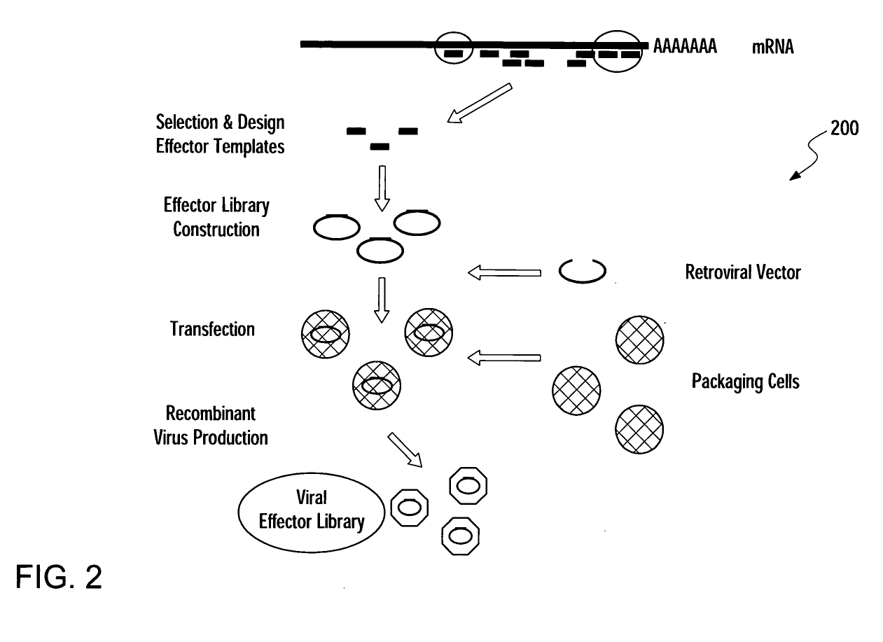

FIG. 2 shows the details of step 200, effector library construction, of method 100 of FIG. 1. In FIG. 2, first, a target, such as an mRNA is selected, and effector nucleic acids are designed. For example, if the effectors are siRNAs, an mRNA target is selected, and various siRNA sequences are selected and constructs are designed. Next, the effector nucleic acids are combined with a vector, preferably a retroviral vector, and more preferably, a lentiviral vector. Once the vector/effector constructs are synthesized and purified, the constructs are used to transfect a packaging cell line. Packaging cells containing the vector/effector constructs generate the effector library.

The viral effector constructs according to the present invention can be generated synthetically or enzymatically by a number of different protocols known to those of skill in the art, and the appropriate oligonucleotide and polynucleotide constructs may be purified using standard recombinant DNA techniques as described in, for example, Sambrook et al., Molecular Cloning: A Laboratory Manual, 2nd Ed., Cold Spring Harbor Press, Cold Spring Harbor, N.Y. (1989), and under regulations described in, e.g., United States Dept. of HHS, National Institute of Health (NIH) Guidelines for Recombinant DNA Research. In a preferred embodiment, the effector constructs are synthesized synthetically using phorphoramidite chemistry and, more preferably, are synthesized on a microarray glass surface or other support surface using photolithography, ink-jet deposition, electrochemical means or other protocols well known in the art.

The vector and other nucleic acid constructs comprising the effector sequences may be used for a number of applications, including effector drug delivery, gene therapy, gene functional analyses, etc. Viral and non-viral vectors may be prepared and used. The choice of vector will depend on the type of cell in which propagation is desired and the purpose of propagation. Certain vectors are useful for amplifying and making large amounts of the desired effector sequence. Other vectors are suitable for expression in cells in culture. The choice of appropriate vector is well within the skill of the art, and many such vectors are available commercially. To prepare the constructs, the effector nucleic acid is inserted into a vector typically by means of DNA ligase attachment to a cleaved restriction enzyme site in a vector or by a protocol known in the art, including use of recombination enzymes, in-fusion PCR, etc.

The vectors used for high efficiency transduction or transfer and expression of the effectors in various cell types are derived preferably from retroviruses. Retroviruses are any virus belonging to the family Retroviridae, comprising single-stranded RNA animal viruses characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

Lentiviruses are members of the retrovirus family. Lentivirus vectors are often pseudotyped with VSV-G, and have been derived from the human immunodeficiency virus (HIV), the etiologic agent of the human acquired immunodeficiency syndrome (AIDS); visan-maedi, which causes encephalitis (visna) or pneumonia in sheep; the caprine arthritis-encephalitis virus, which causes immune deficiency, arthritis, and encephalopathy in goats; equine infectious anemia virus (EIAV), which causes autoimmune hemolytic anemia and encephalopathy in horses; feline immunodeficiency virus (FIV), which causes immune deficiency in cats; bovine immune deficiency virus (BIV) which causes lymphadenopathy and lymphocytosis in cattle; and simian immunodeficiency virus (SIV), which causes immune deficiency and encephalopathy in non-human primates. Vectors that are based on HIV retain <5% of the parental genome, and <25% of the genome is incorporated into packaging constructs, which minimizes the possibility of the generation of revertant replication-competent HIV. Biosafety has been further increased by the development of self-inactivating vectors that contain deletions of the regulatory elements in the downstream long-terminal-repeat sequence, eliminating transcription of the packaging signal that is required for vector mobilization.

Reverse transcription of the retroviral RNA genome occurs in the cytoplasm. Unlike C-type retroviruses, the lentiviral cDNA complexed with other viral factors—known as the pre-initiation complex—is able to translocate across the nuclear membrane and transduce non-dividing cells. A structural feature of the viral cDNA—a DNA flap—seems to contribute to efficient nuclear import. This flap is dependent on the integrity of a central polypurine tract (cPPT) that is located in the viral polymerase gene, so most HIV-1-derived vectors retain this sequence. A recent study has, however, indicated that the presence of a valine residue at position 165 in the viral integrase is more important for the nuclear import of viral nucleic acids than the cPPT. Lentiviruses have broad tropism, low inflammatory potential, and result in an integrated vector. The main limitations are that integration might induce oncogenesis in some applications. The main advantage to the use of lentiviral vectors is that gene transfer is persistent in most tissues or cell types.

The viral construct is a nucleotide sequence that comprises sequences necessary for the production of recombinant retrovirus in a packaging cell and expression of effector molecules, reporters or other genes. Generation of the viral construct can be accomplished using any suitable genetic engineering techniques well known in the art, including without limitation, the standard techniques of PCR, oligonucleotide synthesis, restriction endonuclease digestion, ligation, transformation, plasmid purification, and DNA sequencing. The viral construct may incorporate sequences from the genome of any known organism. In a preferred embodiment, the sequences are derived from mammalian species. The sequences may be incorporated in their native form or may be modified. For example, the sequences may comprise insertions, deletions or substitutions. In a preferred embodiment, the viral construct comprises sequences from a lentivirus genome, such as the FIV genome, and the effector (heterologous) sequence inserts are based on sequences derived from mammals.

The viral construct preferably comprises sequences form the 5′ and 3′ LTRs of a lentivirus. More preferably the viral construct comprises the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. Preferably the LTR sequences are FIV LTR sequences. The virus also can incorporate sequences for MMLV or MSCV, RSV or mammalian genes.

The viral construct preferably comprises an inactivated or self-inactivating 3′ LTR. The 3′ LTR may be made self-inactivating by any method known in the art. In the preferred embodiment the U3 element of the 3′ LTR contains a deletion of its enhancer sequence, preferably the TATA box, Sp1 and NF-kappa B sites. As a result of the self-inactivating 3′ LTR, the provirus that is integrated into the host ell genome will comprise an inactivated 5′ LTR.

Optionally, the U3 sequence from the lentiviral 5′ LTR may be replaced with a promoter sequence in the viral construct. This may increase the titer of virus recovered from the packaging cell line. An enhancer sequence may also be included.

The viral construct is preferably cloned into a plasmid that may be transfected into a packaging cell line. The preferred plasmid preferably comprises sequences useful for replication of the plasmid in bacteria.

The viral construct comprises at least one effector sequence. The effector sequence may be any nucleotide sequence, which optionally is also included on a microarray. Preferably, the effector sequence is based on a sequence of mammalian origin. In one embodiment, the effector sequence comprises at least one RNA coding region. Preferably, in such an embodiment, the RNA coding region is a DNA sequence that can serve as a template for the expression of a desired effector molecule in the host cell.

The viral construct also may comprise one or more effector cassettes under control of RNA Polymerase II or III promoters for expression of the effector molecule(s). In the cassette, the RNA Polymerase promoter is operably linked to the effector region which also may be linked to a termination sequence thus forming an effector cassette for cloning and expression of the effector molecules. In addition, more than one RNA Polymerase promoter may be used to express effector molecules. An “internal” cassette is promoter/enhancer that is located between the 5′ LTR and the 3′ LTR sequences in the viral construct and is operably linked to the effector. In another embodiment, the effector cassette is located in the 3′ LTR or, optionally, in the U3 the region of 3′ LTR. In another embodiment, the internal region further may include a drug-resistance cassette (conferring resistance to Hprt, Neo, or Hyg, for example) or a reporter cassette (coding for fluorescent GFP, RFP, or cell surface enzymes such as luciferase or beta-galactosidase, CD81, for example) under control of a constitutive or regulated promoter.

The promoter/enhancer for the effector, drug-resistance or reporter cassette preferably is selected based on the desired expression pattern of the effector and the specific properties of known promoters/enhancers. Thus, the promoter may be a constituitive promoter, such as the promoter for ubiquitin, CMV, β-actin, histone H4, EF-1alfa or pgk controlled by RNA polymerase II, or the U6 snRNA, H1 snRNA, or tRNA promoters controlled by RNA polymerase III. Alternatively, the promoter may be a tissue-specific promoter such as Ick, mygenin, or thy1 . In addition, promoters may be selected to allow for inducible expression of the effector. A number of systems for the inducible expression using such a promoter are known in the art, including the tetracycline responsive system and the lac operator-repressor system. In one embodiment, opposing promoters (attached both at the 5′ end of a sense strand and at the 5′ end of an antisense strand of an siRNA) is used (see WO 03/022052 A1; and US 2002/0162126 A1).

An enhancer also may be present in the viral construct to increase expression of the gene of interest. For example, a CMV enhancer may be used in combination with the chicken β-actin promoter.

In preferred embodiments of the invention, an effector expression cassette comprises a Pol III promoter and an effector coding region. The effector coding region preferably encodes an effector molecule that down-regulates the expression level of a particular mRNA, protein or proteins. The effector molecule encoded can, for example, be an siRNA-that is, a double-stranded RNA complex, or an RNA molecule having a stem-loop or a so-called “hairpin” structure that inhibits gene expression of genes having an mRNA sequence complementary to one strand of the double-stranded RNA complex through a process termed RNA interference. The duplex portion of the RNA is substantially identical to a sequence of the target gene to be down regulated, and typically is about 15 to about 30 nucleotides in length.

In the case of siRNA, the duplex RNA can be expressed in a cell from a single retroviral construct, preferably, a lentiviral construct. In one embodiment, a single RNA coding region in the construct comprises a sense region, a loop region, and an antisense region. Thus, typically, an siRNA effector cassette would have an RNA Pol III promoter operatively linked to an RNA coding region, which includes a sense region, a loop or hairpin region, and an antisense region. The sense and antisense regions are each about 15 to 30 nucleotides in length, and the loop region is preferably about 2 to 15 nucleotides in length. Once expressed, the sense and antisense portions will form a duplex with a loop at one end.

In another embodiment of an siRNA effector construct, the retroviral construct comprises two RNA coding regions. The first coding region is a template for the expression of a first RNA and the second coding region is for the expression of a second RNA. Following expression, the first and second RNAs form a duplex. This construct also comprises a first Pol III promoter operably linked to the first RNA coding region, and a second Pol III promoter operably linked to the second RNA region. Each coding region may be flanked on the 3′ end by a terminator sequence.

In yet another embodiment, the retroviral construct comprises a first RNA Pol III promoter operably linked to a first RNA coding region, and a second RNA Pol III promoter operably linked to the same first RNA coding region on the opposite strand and in the opposite direction, such that expression of the RNA coding region from the first RNA Pol III promoter results in a synthesis of a first RNA molecule as the sense strand and expression of the RNA coding region from the second RNA Pol III promoter results in synthesis of a second RNA molecule as an antisense strand that is substantially complementary to the first RNA molecule.

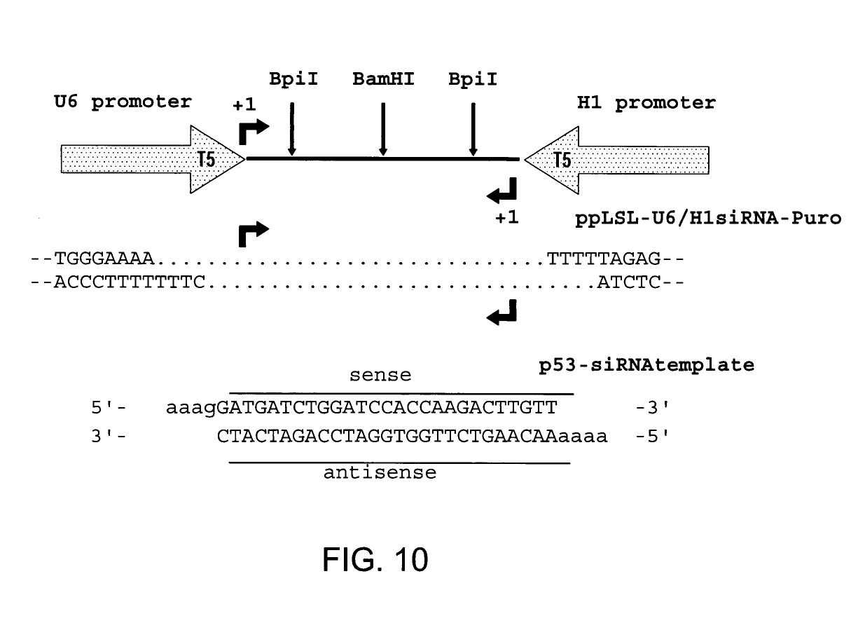

In one such embodiment, each RNA Pol III promoter includes a termination sequence just upstream of the transcription initiation start site, preferably a termination sequence having five consecutive T residues (for example, T5 of FIG. 10). Thus, on one strand, the Pol III promoter is linked to a first terminator sequence immediately before the transcription start site and an RNA coding region (see FIG. 10). Also in this embodiment, the expression cassette has a second Pol III promoter comprising a second terminator linked to a second coding region (substantially the complement of the first coding region). Thus, the siRNA coding sequence in such an embodiment is located, just downstream of both transcriptional start sites, without significant additional non-siRNA sequences. Alternatively, in the case of siRNA, the sense and antisense strands of the RNA duplex can be expressed using two or more retroviral constructs.

As stated, the RNA coding region of an siRNA effector construct may be operatively linked to a terminator sequence. The pol III terminators preferably comprise stretches of four or more thymidine residues. Preferably, a sequence of five consecutive adenines is linked immediately upstream of the RNA coding regions to serve as a terminator for the opposing promoters.

The viral construct also may contain additional genetic elements. The types of elements that may be included in the construct are not limited in any way and may be chosen by one with skill in the art. For example, a reporter gene may be placed in functional relationship with the internal promoter, such as the gene for a fluorescent marker protein. If a marker gene is included along with the effector, an internal ribosomal entry site (IRES) sequence can be included. Alternatively, the additional genetic elements can be operably linked with and controlled by an independent promoter/enhancer.

Any method known in the art may be used to produce infectious retroviral particles whose genome comprises an RNA copy of the viral construct described herein. Preferably, the viral construct is introduced into a packaging cell line. The packaging cell line provides the viral proteins that are required in trans for the packaging of the viral genomic RNA into viral particles. The packaging cell line may be any cell line that is capable of expressing retroviral proteins, including 293, HeLa, D17, MDCK, BHK, and Cf2Th.

In some embodiments, the effector construct is used together with a viral reporter construct which may comprise one or more reporter genes under the control of a constitutive or conditional (regulatable) promoter. In a preferred embodiment, at least one of the reporter genes is controlled by a signaling pathway-specific promoter (conditional) and a second reporter gene is controlled by a constitutive promoter.

The packaging cell line may stably express necessary viral proteins. Such a packaging cell line is described, for example, in U.S. Pat. No. 6,218,181.

Alternatively, a packaging cell line may be transiently transfected with plasmids comprising nucleic acids that encode the necessary viral proteins. In another embodiment, a packaging cell line that does not stably express the necessary viral proteins is co-transfected with two or more plasmids. One of the plasmids comprises the viral construct comprising the effector. The other plasmid(s) comprises nucleic acids encoding the proteins necessary to allow the cells to produce functional virus that is able to infect the desired host cell.

The packaging cell line may not express envelope gene products. In this case, the packaging cell line will package the viral genome into particles that lack an envelope protein. As the envelope protein is responsible, in part, for the host range of the viral particles, the viruses preferably are pseudotyped. A “pseudotyped” retrovirus is a retroviral particle having an envelope protein that is from a virus other than the virus from which the RNA genome is derived. The envelope protein may be from a different retrovirus or a non-retrovirus. One envelope protein is the vesicular stomatitius virus G (VSV-G) protein. Thus, the packaging cell line preferably is transfected with a plasmid comprising sequences encoding a membrane-associated protein, such as VSV-G, that will permit entry of the virus into a host cell. One with skill in the art can choose an appropriate pseudotype for the host cell used. In addition to conferring a specific host range, a chosen pseudotype may permit the virus to be concentrated to a very high titer. Viruses alternatively can be pseudotyped with ecotropic envelope proteins that limit infection to a specific species.

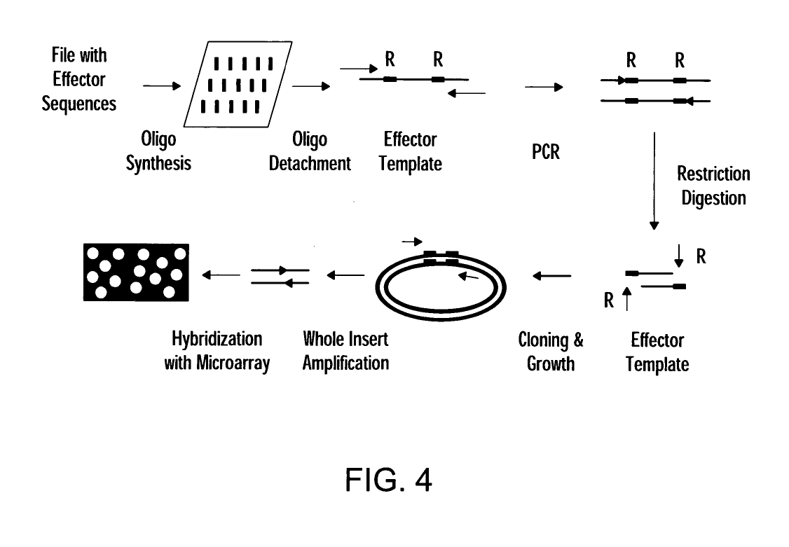

FIG. 4 shows an optional, additional quality control aspect of the formation of the viral effector library shown in FIG. 2. In FIG. 4, a computer file with the selected effector sequences is used to synthesize oligonucleotides (effector templates) on, for example, duplicate or triplicate microarrays. The effector templates are detached from one of the arrays, and are amplified by PCR. Alternatively, the extension or PCR step can be performed directly on a chip or oligonucleotides can be used directly for generating effector templates. After amplification, the now double-stranded effector templates are digested with a restriction enzyme to produce a sticky-ended double stranded effector template, which can be inserted into an appropriate vector (such as a retroviral or lentiviral vector as described herein) that has been linearized by, preferably, the same restriction enzyme. Once the effector templates have been inserted into the chosen vector, whole insert amplification can be performed and the amplified inserts can be hybridized to one of the corresponding microarrays to verify representation of all effector sequences. Such techniques and alternative techniques are well known to those skilled in the art.

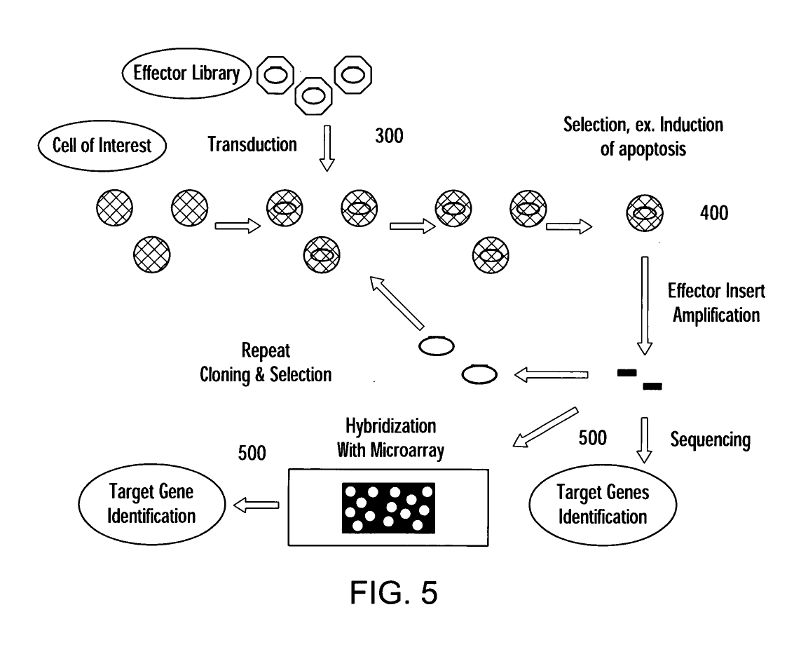

FIG. 5 is a schematic of a general design of method 100, beginning with the effector library (the steps for the construction of which are illustrated in FIG. 2). FIG. 5 shows the steps of transduction of the target cells or cells of interest (step 300), selection of target cells with a desired phenotype (step 400), and effector insert amplification and effector identification (step 500). Effector identification analysis may be accomplished in various methods known in the art, including, for example, sequencing, or, preferably, hybridization to a microarray. Alternatively, the amplified inserts can be re-cloned into vectors, transfected into packaging cells, and used to transduce additional target cells. The effector insert amplification step may be replaced by a virus rescue technique by cloning viral sequences from genomic DNA into plasmids suitable for introduction into E. coli.

Transduction of the target cells with the pre-packaged viral effector library may be accomplished by methods known by those skilled in the art and depends generally on the target cell type and the viral vectors employed (step 300 of FIG. 1 and seen in FIGS. 5, 6 and 7). The target cells can be a pure, homogeneous population of the same or similar cells or the target cells can be a heterogeneous population of different cell types. The target cells may be cultured, or may be tissues, organs, biological fluids or whole organisms. Preferably, the organism is a human, mouse or rat. The effector library may be co-transduced with a reporter vector in order to extend selection of target cells to a variety of in vivo and in vitro biological assays.

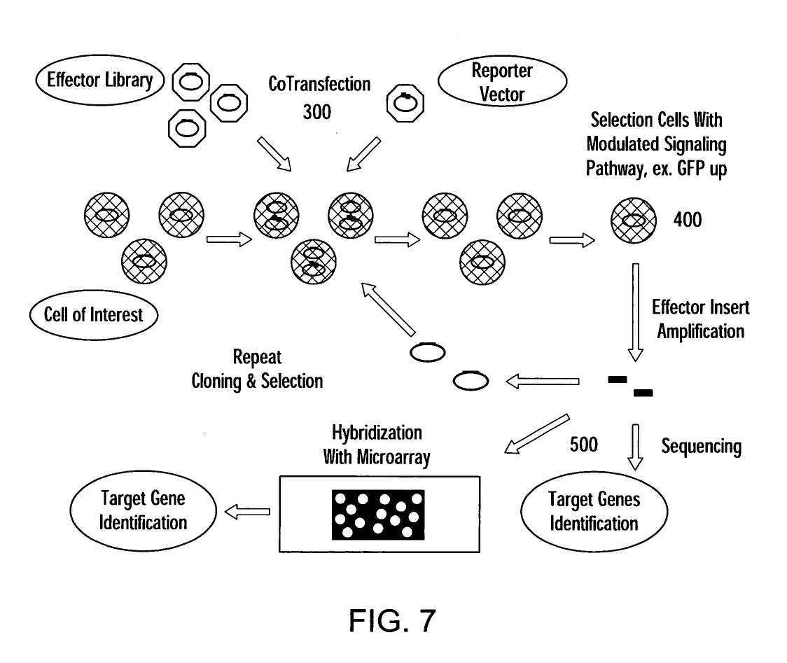

Once transduced, the target cells must be assayed for a particular phenotype (step 400 of FIG. 1 and seen in FIGS. 5 and 7). To date, the most frequently used selection strategy in genetic screens with effector libraries in mammalian cells has been improved cell survival after exposure to cytotoxic drugs or apoptogenic agents, like resistance to the DNA damaging drug cisplatin in mouse embryo fibroblasts (Erez, N., et al., Oncogene, 21:6713-21 (2002)), resistance to TNF-induced apoptosis in mouse NIH 3T3 cells (Nezanov, N., et al., J. Biol. Chem., 278:3809-15 (2003)), and resistance to topoisomerase 11 inhibitor 9-OH-ellipticine in DC-3F cells (see, e.g., Cross, L., et al., Cancer Res., 63:164-71 (2003)). In another embodiment of the present invention, cells of a desired phenotype are selected by the selective binding to an agent, such as a low molecular weight entitiy such as a drug, amino acid, sugar, or nucleotide, or a high molecular weight entity such as a polysaccharide, polynucleotide, cytokine, antibody, matrix, bead or the like. In another embodiment, the cells after treatment can be analyzed directly without the selection (isolation) step.

One of the most flexible methods of selection, particularly suitable to the isolation of effectors with modest phenotypic effects, is based on the use of fluorescence-activated cell sorting (FACS) to separate cell populations with quantitatively different levels of phenotype-specific proteins detectable by immunofluorescent staining or other fluorescent reporters. Additional flexibility in selection strategy/assay design can be achieved with construction of cell lines expressing a fluorescent reporter protein that reflects the activation of a specific promoter controlled by a specific transcriptional factor. This approach allows identification of complex components (proteins) in a signaling pathway that affects activity of downstream transcription factors.

Reporter cell lines are already commonly used for ultra-high throughput screening of chemical libraries in drug discovery, as the majority of drugs function through molecular targets operating in signal transduction pathways. Genetic screens combined with use of transcriptional reporter cell lines and effector libraries have been applied successfully to identify proteins, their inhibitors and regulators of IL-4, TGF-beta, and myc-dependent pathways (see, e.g., Rodriguez, C., et al., J. Bio. Chem., 276:30224-230 (2001); Lorens, J. B., et al., Curr. Opin, Biotechnol., 12:613-21 (2001); and Nikiforov, M.A., et al., Oncogene, 19:4828-31 (2000)). Use of a genetic screen based on lentiviral reporter vectors and prepackaged effector libraries in the pseudotyped particles extends significantly the application of this technology to analysis of signal transduction pathways in primary cell lines, stem cells, cells isolated from organisms (blood cells) or even directly in model organisms (mouse). Lentiviral-based vectors provide allow cotransduction and expression of effector and reporter constructs in the same cell, avoiding complicated construction of disease-specific reporter cell lines prior to gene functional analysis.

Moreover, in certain embodiments, a set of dual fluorescent reporter lentiviral vectors may be used for FACS-based genetic screens. Such transcriptional reporter vectors optionally comprise expression cassettes designed to measure accurately levels of, for example, a GFP reporter under the transcriptional control of pathway-specific promoter. Such vectors also may include an additional transcription unit used for a normalization control, such as, for example, expression of an RFP reporter under control of a constitutive promoter, for example, that derived from the histone H4 gene. FACS may then be employed to detect changes in the ratio of GFP/RFP fluorescence in transduced cells, which reflects perturbation in the mechanism of specific pathway induced by the effector. The transcriptional reporter vectors described here can be used for functional studies in human, mouse and rat systems, as pathway-specific response element sequences in promoter regions have been found to be highly conserved amongst mammalian species.

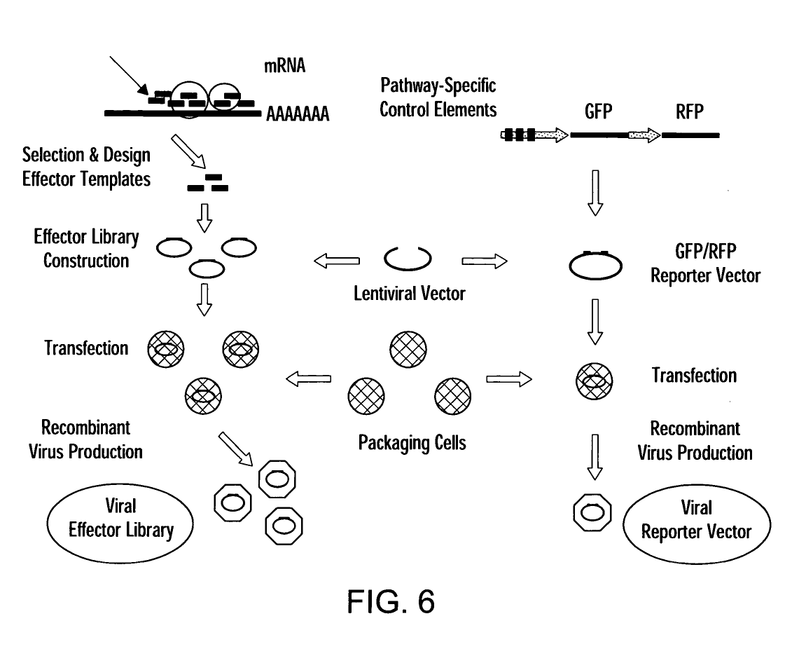

FIG. 6 shows essentially the same sequence of steps as that of FIG. 2; however FIG. 6 show additionally the parallel synthesis of a dual reporter viral vector construct contained within a viral particle. Here, the dual reporter vector contains GFP, under transcriptional control of a pathway-specific promoter, and RFP, under transcriptional control of a constituitive promoter. Both the dual reporter cassette and the effector template are ligated into a lentiviral vector, and both as transfected into packaging cells. The effector constructs form an effector library, and the reporter vectors form a reporter virus.

The use of lentiviral vectors for delivery of the reporter cassettes represents a significant improvement in the generation of various reporter cell lines. The approach utilizes the natural ability of the virus genome to integrate into transcriptionally active sites of the host genome. Unlike random integration following DNA transfection that frequently generates rearrangements and tandem amplifications, the virally-transduced cassettes uniformly are integrated as single intact inserts surrounded by viral LTRs that possess certain barrier functions.

The efficiency of lentiviral infection at virtually 100% of dividing and resting cells allows introduction of reporter cassettes efficiently and without any selection. Most importantly, the uniformity of expression from different retroviral inserts and lack of significant position effect allows working with polyclonal populations of cells while avoiding the problem of clonal variability. In addition, the approach can be applied to the cells within a living organism, as the reporter cassettes easily can be introduced by injection or other delivery means of the virus into different organs, tumors, or embryos. Expansion of signaling pathway-specific lentiviral-based reporter vectors and reporter cell lines based on these vectors designed for functional screens is an important step toward versatile high throughput functional genomics.

FIG. 7 is similar to FIG. 5, and picks up where FIG. 6 leaves off. FIG. 7 shows the steps of transduction of target cells (step 300), here with both the effector library and the dual reporter vector construct, selection of the desired phenotype (shown by a change in GFP expression/GFP fluorescence intensity) (step 400), and effector identification (step 500).

The preferred method of effector identification (step 500 of FIG. 1 and seen in FIGS. 5 and 7) of the present method is by microarray. Preferably, the effector sequences included in the viral library are those probe sequences that are included on the microarray or microarrays used for analysis. Briefly, a microarray is an ordered arrangement (defined location) of probe, typically nucleic acids, on a solid or semi-solid support. In some embodiments, there are more than 35,000 such probe biomolecules comprising probe nucleic acids. Thus, if a target cell is transduced with one or more of the effector molecules and a selectable phenotype results, the effector nucleic acids can be isolated from selected cells and analyzed by hybridization to a microarray. In some embodiments, target cells are assayed before being transduced with the effector library, then are assayed after being transduced with an effector library and after subsequent treatment with an experimental compound. The results of microarray analysis for the pre-transduced cells can be compared with the results of the microarray analysis for the transduced and treated target cells, and the proper effectors with specific effect may be identified.

Using the same sequences for the siRNA effector and the microarray will permit use of the microarray for high-throughput identification of the siRNA effectors that are involved in or modulate specific pathways or are responsible for specific cellular phenotype. Such cellular phenotypes include drug resistance; susceptibility to apoptosis, infection, radiation, or stress, etc.; uncontrolled proliferation or migration; expression of specific markers; or interaction with other cells or substrates, etc.

There are many microarrays and microarray vendors known in the art. For example, Affymetrix, Inc. (Santa Clara, Calif.), manufactures the GeneChip® by in situ photolithographic synthesis of approximately 10,000 to 260,000 25-mer oligonucleotides onto silicon wafers, which are then diced into chips. The readout or analysis is accomplished by fluorescence. Agilent, BDB Clontech, Amersham, and ABI provide about 10,000 500-5000-mer cDNAs or 4,000—35,000 45-80-mer oligonucleotides printed by ink-jet printer or by pins onto glass slides, which are also analyzed by fluorescence, radioactivity or chemiluminescense. Essentially, microarrays can be made by photolithography, spotting oligonucleotides synthesized by standard phosphoramidite chemistry, photochemistry, electrochemistry, or the like. Analysis techniques include fluorescence, mass spectrometry, chemiluminescense or radioisotopic methods.

### EXAMPLE 1

### Development of Lentiviral Vectors for Cloning Effector Libraries.

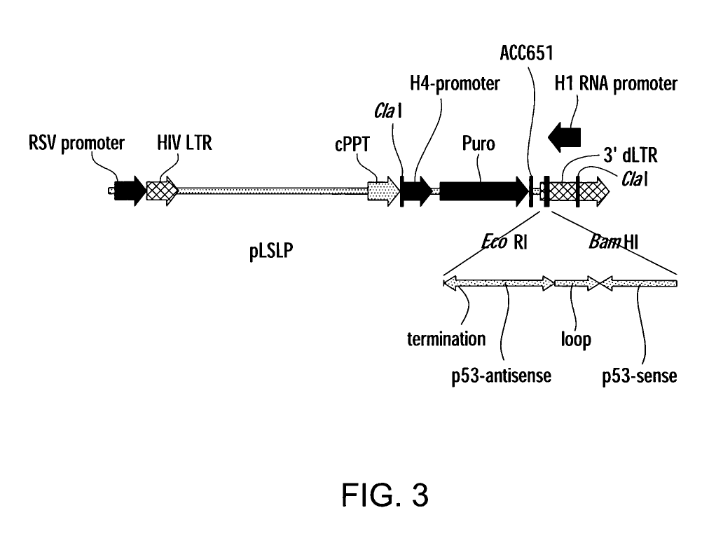

In order to develop an efficient siRNA library transduction vector; a pL-reporter lentiviral backbone was used in which the puromycin-resistance gene is controlled by H4 promoter (see FIG. 3). A small polylinker containing Clal, BamHI and EcoRI restriction nuclease sites was introduced into the U3 region of the 3′ LTR. The polylinker can be used to clone single promoter (H1) siRNA expression cassette or double promoter (U6/H1) siRNA expression cassette.

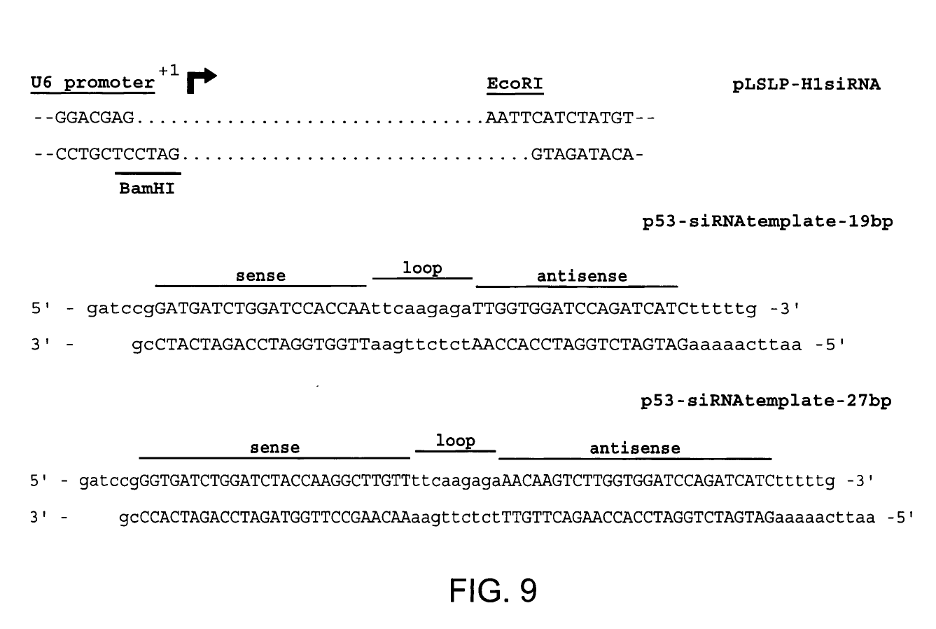

In one embodiment, a single promoter H1siRNA expression cassette was assembled from chemically synthesized oligonucleotides and cloned into the Clal-BamHI sites to direct transcription of small hairpin RNAs, which are processed into functional siRNA by cellular enzymes. The resulting single promoter expression cassette pLSLP construct was very similar in design to other retroviral vectors that have been used successfully for cloning and expression of siRNA (see, e.g., Qin, X. F., et al. PNAS, 100:183-88 (2003); Tiscorna, G., et al., PNAS, 100:1844-48 (2003); and Xia, H., et al. Nature Biotech, 20:1006-10 (2002)). [0078] Two exemplary effector constructs have been made and cloned into the pLSLP-H1siRNA vector (see FIG. 9). The constructs in FIG. 9 differ in the length of the siRNA inserts. In one case, the siRNA component is 19 bp in length, in the other case, the siRNA is 27 bp in length. The duplex at the top of the Figure shows the sequence of the lentiviral vector after digestion with BamH1 and EcoRI. The dotted lines indicate the position of the removed nucleotides (a stuffer sequence used in the vector as a placeholder until the effector construct is inserted), the dashed lines indicate the continuing sequence of the vector, and +1 indicates the position of the transcription start site, and the string of Ts indicate the termination sequence.

A pLSLP-H1siRNA vector small hairpin RNA can be transcribed from the RNA-polymerase III-driven siRNA template construct and cloned by ligation into the unique BamHI and EcoRI sites. The two siRNA constructs that target p53 mRNA; namely, the 19 bp hairpin and a 27 bp hairpin construct (with a 9-nucleotide loop), were tested and compared using a pL-p53RE-LacZ-H4-puro bearing HeLa reporter cell line (details of this HeLa line are presented in Example 3, infra).

In addition, the HeLa reporter cell line carrying the pL-p53RE-LacZ-H4-puro construct was transfected by lipofectamine with the two p53 mRNA pLSLP-p53siRNA plasmids. The pLSLP vector and p53-siRNA constructs with previously confirmed activity were used as negative and positive controls. Thirty-six hours after lipofection, the cells were treated for 18 hours with the drug PRES-229 (2 μg/ml) that activates transcription of p53 in HeLa cells, and beta-galactosidase was measured in a 2 hour ONPG reaction. Results demonstrated that, on average, the 27 bp hairpin siRNA constructs were more efficient than the 19 bp constructs in silencing p53. About 50% of the 27 bp siRNAs designed from the p53 sequences demonstrate at least 80% silencing efficiency for p53.

In another embodiment, the double promoter (U6/H1) cassette was developed and cloned into the pLSLP vector. FIG. 10 illustrates the design of a dual promoter cassette. In this cassette, both the H1 and U6 promoters have modified sequences and incorporate a polymerase III transcriptional termination signal AAAAA/TTTTT just upstream of the transcriptional start sites. The stuffer fragment inserted between transcriptional start sites has two unique Bpil sites. Bpil restrictase digests DNA outside of the recognition sequence, thus allowing the insertion of the effector cassette (here, siRNA) at +1 position of the promoter sequence. The opposing promoter cassette allows cloning of siRNA templates assembled from a short sense oligonucleotide and an antisense oligonucleotide without a stem-loop structure. As a result of in vivo transcription, the resulting siRNA has a double-stranded structure without additional non-mRNA sequences, with the exception of the initiation nucleotide (G) and a (U)2-3 overhang at the 3′ ends.

The double-stranded siRNA constructs were found to provide similar silencing efficiency similar to hairpin-type siRNA constructs designed for the same 27-bp p53 sequence and tested in the luciferase assay as described above. Replacement of the wild-type promoter sequences with a termination signal just upstream of the transcriptional start site was found not to change activity of the promoters. The dual promoter construct has been found to be more stable than the stem-loop construct during the propagation in E. coli and mammalian cells.

In another embodiment, the siRNA library vector was based on a biologically safe FIV viral vector developed at UCSD ( U.S. Pat. No. 6,555,107 B2). One reason for the selection of a non-primate FIV-based vector system and development of FIV-based products is due to safety. The pFIV vectors have a structure very similar to pL-based vectors just described. Specific blocks of the FIV genome were used to replace the corresponding HIV elements in the pLSLP vectors.

The resulting pFIV siRNA cloning vectors have several important features. First, the FIV siRNA vector contains a hybrid RSV/FIV promoter in the 5′ -LTR with lentiviral R and U5 sequences for efficient control of vector packaging and integration. Also, the FIV siRNA vector contains a deletion in the enhancer of the U3 region of 3′ -LTR to provide a self-inactivating property to the reporter vector. In addition, the pFIV siRNA cloning vector contains the region of the FIV genome required for efficient packaging: 1) a central purine pyrimidine tract (cPPT) derived from the FIV genome that is important for improved nuclear translocation in non-dividing cells (which increases the efficiency of gene transfer); 2) internal expression cassettes controlling fluorescent reporter protein or drug resistance gene flanked by unique restriction sites; and 3) unique restriction sites providing a modular structure (facilitating interchangeable replacement of reporter genes, drug resistance genes, transcription factor response elements and minimal promoters).

In another embodiment, several siRNA library cloning vectors were designed so that the siRNA expression cassette may be controlled by an RNA-polymerase III promoter under the regulation of small molecules like tetracycline. FIG. 10 is a schematic representation of constructs with repression (A) and activation (B) of siRNA expression. (A) In the absence of tetracycline, the dimerized tetracycine repressor (Tet R) binds to the tetracycline operator sequence (tet 0) located between the proximal sequence element (PSE) and the TATA box (TATA). This complex interferes with the formation of the transcription initiation complex, which consists of the snRNA activating protein complex (SNAPc), the transcription factor IIIB (TFIIIB) and RNA polymerase III (Pol III). (B) In the presence of tetracycline, the IIIB (TFIIIB) and RNA polymerase III (Pol III). (B) In the presence of tetracycline, the complex can position itself properly.

### EXAMPLE 2

### Construction of Effector Libraries in Lentiviral Vectors and Testing Effector Representation in Effector Libraries

A protocol for the construction of high complexity siRNA libraries in single promoter and double promoter pLSLP vectors was developed, as was a test for the stability of these siRNA libraries by hybridization with the Affymetrix Human Genome Focus Array (Affymetrix Cat. N 900377). The Affymetrix Human Genome Focus Array comprises about 90,000 (25-nucleotide long) oligonucleotide probes for 8,500 unique human transcripts (using about 11 probes/sequence) derived from RefSeq database. Based on genes present in the human Genome Focus Array, 1,500 genes related to cancer were selected for siRNA library construction. Five siRNA template oligos (27-mers) were designed for each of these genes based on selection parameters that have been developed for choosing efficient siRNAs. The 7,500 oligos (5×1,500 genes) were synthesized on the surface of glass slides (custom 7.8K “Xeotron” chips) for each single and double promoter vector comprising the sequences of the array probe oligos flanked by conservative sequences with restriction sites at both the 5′- and 3′-ends (FIG. 13).

In order to make the siRNA libraries, oligos were detached from the glass surface by alkaline treatment, the siRNA templates were amplified by PCR using flanking primers, and the PCR products were digested by restriction enzymes and cloned into a linearized single promoter vector (pLSL-H1siRNA-Puro) or a linearized double promoter vector (pLSL-U6/H1 siRNA-Puro).

After ligation, the siRNA library was transfected into competent DH5-alfa cells, grown as independent colonies on LB agar plates, and total siRNA library DNA was purified from a pool of about 100,000 independent ampicilin-resistant bacterial colonies.

In order to study the representation (stability) of siRNA inserts in the siRNA libraries, siRNA template inserts were amplified from the siRNA library DNA using biotinilated vector primers. Ten 10 ug of PCR product was hybridized with the Affymetrix Human Genome Focus Array using the manufacturer's protocol. As a positive control, siRNA template DNA was amplified from the mixture of the 7,500 oligos detached from the surface of the Xeotron Chip using biotinylated flanking primer. These amplified products were then hybridized to an Affymetrix Human Genome Focus array. Intensity signals were analyzed in the positions of the array corresponding to the locations of the oligonucleotides that were used for siRNA library construction.

Overall, the siRNA library generated in the single promoter vector (hairpin structured siRNA, FIG. 9) demonstrated rather low stability, with loss of about 50% of sequences during amplification and cloning steps (determined by measuring a reduction in signal intensity more than 5-fold). The siRNA library generated in the double promoter vector (pLSL-U6/H1 siRNA-Puro) (double-stranded siRNA with no loop structure, FIG. 10) demonstrated good stability (at least 90%) of siRNA inserts using the same 5-fold cut-off criteria. Sequence analysis of randomly selected clones revealed that more than 95% of the clones from both libraries had correct inserts with a mutation rate of about 1% (1 mutation in 100 nucleotides).

### EXAMPLE 3

### Development of Lentiviral Transcriptional Reporter Vectors and their us with Eff ctor Constructs.

A novel HIV-based lentiviral transcriptional reporter vector, pL-p53RE-LacZ-H4-puro, has been developed. This reporter vector was originally designed to screen for chemical compounds that might activate a p53-dependent pathway using induction of beta-galactosidase as a reporter. The lentiviral backbone of the pL-p53RE-LacZ-H4-puro vector was derived from a self-inactivating lentiviral vector pLV-GFP (Pfeifer, et al., PNAS, 99: 2140 (2002)). The pL-p53RE-LacZ-H4-puro vector was engineered to provide high viral titer and to inactivate the promoter in the 5′ LTR following integration into the host genome. Self-inactivating vectors provide more consistent and improved expression due to reduced promoter interference, and self-inactivating lentiviral vectors are safer to work with because they are less likely to form replication-competent retrovirus.

In order to construct the pL-p53RE-LacZ-H4-puro vector, a synthetic multiple cloning site (MCS) was inserted into a basic lentiviral backbone and the following elements were cloned into the MCS: a 20 bp high-affinity consensus p53 binding sequence, a 43 bp “fragment A” from the ribosome gene cluster containing several p53-binding sites, six copies of a p53-binding site from a human gene for the CDK inhibitor p21WAF1/Cip1 (Deiry, et all, Cell, 75:815 (1993)), the minimal immediate early promoter of cytomegalovirus (mCMV), a bacterial LacZ gene encoding beta-galactosidase, and a puromycin-resistance gene under control of constitutive promoter of histone H4. The Xbal-Spel sites were used for directional cloning of various novel pathway-specific response elements. In addition, the Xhol and BamHI sites were used, for example on one embodiment, for the cloning of a destabilized GFP reporter gene. A dsRed reporter gene could replace the puro gene using Nhel-Acc651 sites in the same or yet another embodiment.

In order to test the performance of this reporter vector in gene silencing experiments, the pL-p53RE-LacZ-H4-puro construct was packaged in pseudoviral particles by cotransfection with a pCMV-delta 8.2 plasmid and the pVSV-G plasmid, which express the VSV-G envelope protein in the 293T packaging cell line. After 48 hours, the virus-containing supernatants were collected and used for infection of HeLa cells. A stable p53 transcriptional reporter cell line was generated after selection of puromicine-resistant HeLa cells.

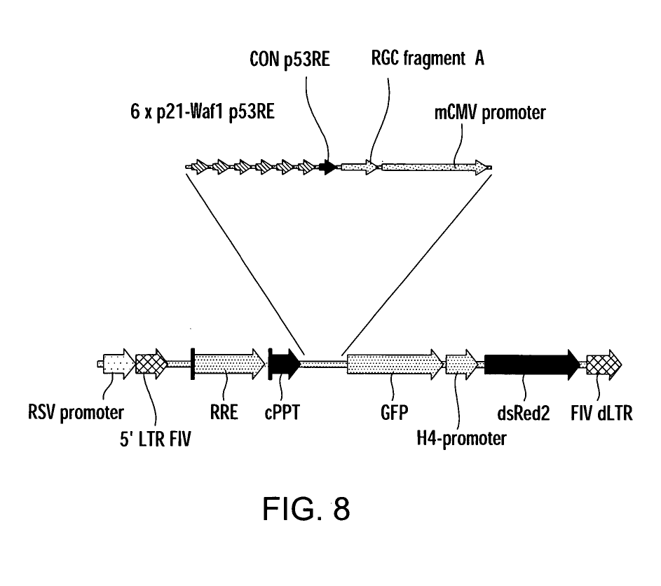

The performance of the dual color fluorescent reporter vectors and a collection of gene-specific siRNA constructs was tested in functional analysis, using a functional assay of the p53 tumor suppressor pathway in normal human embryonic fibroblasts (HEFs) and in an HPV18-positive cervical carcinoma cell line (HeLa) where the activity of p53 is inhibited by binding to the HPV gene product E6. The pFIV-p53RE-GFP/RFP or pL-p53RE-GFP/RFP reporter vector (see, e.g., FIG. 8) and pLSL-p53siRNA construct, which expresses hairpin-type p53 siRNA, were co-transfected into HEFs and HeLa cells. This construct was capable of reducing the level of endogenous p53 protein about 20-fold. The reporter (1×10⁶ infection units) and the siRNA construct (1×10⁶ infection units) prepackaged into viral particles were delivered to 2×10⁶ HeLa (or HEFs) cells using standard protocols (see, e.g., RNA Viruses: A Practical Approach (A. J. Cann, Ed. Oxford University Press (2000)). The efficiency of inhibition of the p53-pathway was then estimated by FACS analysis of the GFP/RFP ratio in HeLa (or HEF) cells at 48 hours and 72 hours post infection. The abundance level of a particular target mRNA in the cells after viral infection is analyzed by PCR using gene-specific primers. This experiment imitates the genetic screen performed with the siRNA libraries and a 20-fold reduction in the ratio of GFP/RFP should be seen.

### EXAMPLE 4

### Identification of Effector Sequences that Effect the Cell Phenotype of Interest.

The lentiviral siRNA library comprising 7,500 siRNAs against 1,500 human mRNAs was constructed in a single promoter pLSLP vector, and packaged in viral particles as described in Examples 2 and 3. The lentiviral siRNA library was then infected into an HCT1 16 p53 −/− colorectal carcinoma cell line with somatic knockout of both alleles of p53. After infection, DNA was isolated from a subpopulation of cells while the rest of the cells were exposed to treatments of gamma irradiation via a Cesium source. Gamma irradiation usually elicits a DNA damage response within these cells, most often a p53 response, resulting in both growth arrest and apoptosis. Since a line that is null for p53 was used, it was expected that a knockdown of one of the genes targeted by the library would suppress the p53 response (including an siRNA against p53). Identification siRNAs that can protect against gamma irradiation would be extremely valuable for drug target development.

The genomic DNA samples were purified from control and gamma-irradiated cells 3 days after exposure and the siRNA inserts were amplified from 5 ug of genomic DNA after two rounds of PCR using biotinilated vector primers. Ten ug of the amplification products were hybridixed with an Affymetrix Human Genome Focus Array using the manufacturer's protocol. All steps of DNA purification and amplification were performed using standard protocols well known in the art.

In order to identify the siRNA inserts and therefore the genes responsible for a gamma-radiation resistant phenotype, intensity signals were compared between control and treated samples in the positions of the array corresponding to the location of oligonucleotides used for siRNA library construction. The comparison analysis revealed that 10 siRNAs--including the p53 siRNA--were significantly enriched at least 10-fold in the treated cell population in comparison with the untreated control population.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material, or process to the objective, spirit and scope of the present invention. All such modifications are intended to be within the scope of the invention.

All references cited herein are to aid in the understanding of the invention, and are incorporated in their entireties for all purposes.

1. A viral transcriptional reporter vector comprising:
a vector backbone derived from a virus of the family Retroviridae;
a conditional promoter and a first reporter cassette under control of the conditional promoter, wherein said transcriptional reporter vector is packaged in viral particles.

2. The viral transcriptional reporter vector of claim 1, wherein the virus is a lentivirus.

3. The viral transcriptional reporter vector of claim 1, further comprising a constitutive promoter and a second reporter cassette under the control of the constitutive promoter, wherein said first and second reporter cassettes generate distinguishable effects in biological assays.

4. The viral transcriptional reporter vector of claim 3, wherein the reporter cassettes generate fluorescent signals, colorimetric signals, or combinations thereof.

5. The viral transcriptional reporter vector of claim 4, wherein the second reporter cassette is an RFP cassette.

6. The viral transcriptional reporter vector of claim 5, wherein the first reporter cassette comprises a coding region selected from the group consisting of beta-galactosidase coding regions or GFP coding regions.

7. The viral transcriptional reporter vector of claim 1, wherein the first and second reporter cassettes comprise coding regions for two different fluorescent proteins.

8. The viral transcriptional reporter vector of claim 7, wherein the two different fluorescent proteins are GFP and RFP.

9. The viral transcriptional reporter vector of claim 1, wherein the retrovirus-derived backbone is FIV-based.

10. The viral transcriptional reporter vector of claim 1, wherein the retrovirus-derived backbone is derived from HIV, visan-maedi, caprine arthritis-encephalitis virus, EIAV, BIV, or SIV.

11. The viral transcriptional reporter vector of claim 1, wherein the constitutive promoter is selected from the group consisting of a histone H4 promoter, a minimal immediately early promoter of cytomegalovirus, a pgk promoter, an EF-1 alfa promoter, and a ubiquitin promoter.

12. The viral transcriptional reporter vector of claim 1, further comprising sequences from the 5′ and 3′ LTRs of a retrovirus.

13. The viral transcriptional reporter vector of claim 1, further comprising a central polypurine tract of a retroviral polymerase gene.

14. A viral transcriptional reporter vector comprising:
a lentivirus-derived vector backbone; and
a conditional promoter comprising one or more pathway-specific response elements and a first reporter cassette under the control of the conditional promoter.

15. The viral transcriptional reporter vector of claim 14, wherein the one or more pathway-specific response elements comprise a p53 binding sequence.

16. The viral transcriptional reporter vector of claim 15, wherein the one or more pathway-specific response elements comprise p53 binding sequences from p21.

17. The viral transcriptional reporter vector of claim 14, wherein the conditional promoter further comprises a minimal immediate early promoter of cytomegalovirus.

18. The viral transcriptional reporter vector of claim 14, further comprising a constitutive promoter and a second reporter cassette under the control of the constitutive promoter.

19. The viral transcriptional reporter vector of claim 18, wherein the reporter cassettes produce calorimetric signals, fluorescent signals, luminescent signals or combinations thereof in cell-based assays.

20. The viral transcriptional reporter vector of claim 18, wherein the first and second reporter cassettes comprise coding regions for two different fluorescent proteins.

21. The viral transcriptional reporter vector of claim 14, wherein the lentivirus-derived backbone is FIV-based.

22. The viral transcriptional reporter vector of claim 14, wherein the lentivirus backbone is derived from HIV, visan-maedi, caprine arthritis-encephalitis virus, EIAV, BIV, and SIV.

23. The viral transcriptional reporter vector of claim 14, further comprising sequences from the 5′ and 3′ LTRs of a lentivirus.

24. The viral transcriptional reporter vector of claim 14, further comprising a central polypurine tract of a lentiviral polymerase gene.

25. A reporter cell generated by transduction of a cell with the packaged viral transcriptional reporter vector of claim 1.

26. The reporter cell of claim 25, wherein the viral reporter vector is stably integrated into the reporter cell's genome.

27. The reporter cell of claim 25, wherein the viral transcriptional reporter vector further comprises a constitutive promoter and a second reporter cassette under the control of the constitutive promoter.

28. A packaged virus comprising:
a viral reporter vector comprising:
a lentivirus-derived vector backbone; a conditional promoter and a first reporter cassette under the control of the conditional promoter; and virion proteins.

29. The packaged virus of claim 28, wherein the viral transcriptional reporter vector further comprises a constitutive promoter and a second reporter cassette under the control of the constitutive promoter.

30. A it comprising:
a viral transcriptional reporter vector comprising:
a lentivirus-derived vector backbone; and
a conditional promoter and a first reporter cassette under the control of the conditional promoter.

31. A kit comprising:
a viral transcriptional reporter vector comprising:
a lentivirus-derived vector backbone; and
a conditional promoter and a first reporter cassette under control of the conditional promoter, wherein said transcriptional reporter vector is packaged in viral particles .

32. The kit of claim 31, further comprising a packaging plasmid which expresses necessary viral proteins for packaging said transcriptional reporter vector into the viral particles in a packaging cell line.

33. The kit of claim 32, wherein the viral reporter vector further comprises a constitutive promoter and a second reporter cassette under the control of the constitutive promoter.
